(19) United States
(12) Patent Application Publication
(10) Pub. No.: US 2021/0011016 A1
(43) Pub. Date: Jan. 14, 2021

(54) ENZYME-AMPLIFIED LATERAL FLOW DEVICE

(71) Applicant: Sumitomo Chemical Company Limited, Tokyo (JP)

(72) Inventor: Jonathan Behrendt, Bedford (GB)

(73) Assignee: Sumitomo Chemical Company Limited, Tokyo (JP)

(21) Appl. No.: 16/978,085

(22) PCT Filed: Feb. 19, 2019
(86) PCT No.: PCT/EP2019/054095

§ 371 Date: Sep. 3, 2020

(30) Foreign Application Priority Data
Mar. 8, 2018 (GB) 1803713

(51) Int. Cl. G01N 33/543; C12N 9/08; C12N 9/04; B01D 69/14; G01N 33/558

(52) U.S. Cl. CPC G01N 33/54386; C12N 9/0065; C12N 9/0006; C12Y 101/03004; G01N 33/558; C12Y 111/01007; B01D 69/141

(57) ABSTRACT

The invention concerns a lateral flow assay device for determining the presence of an analyte in a liquid sample; the use of said device to test for the presence of an analyte in a liquid sample; and a method for determining the presence of an analyte in a liquid sample 5 involving the use of said device.

### FIELD OF THE INVENTION

The invention concerns a lateral flow assay device for determining the presence of an analyte in a liquid sample; the use of said device to test for the presence of an analyte in a liquid sample; and a method for determining the presence of an analyte in a liquid sample involving the use of said device.

### BACKGROUND OF THE INVENTION

The use of testing kits to identify the presence of specific analytes within biological samples such as saliva, blood or urine is known for a variety of purposes, including medical diagnosis, screening and management of long term health conditions. Lateral flow devices (also known as ‘lateral flow immunoassays’) are commonly used for such analyte detection, and examples of such devices include home pregnancy tests, home ovulation tests, and tests for other hormones, pathogens or drugs.

In typical lateral flow test devices, a liquid sample such as saliva, blood or urine is introduced at one end of a porous strip (also known as a ‘lateral flow membrane’), and the sample is then drawn along the strip by capillary action (also known as ‘wicking’). A portion of the lateral flow membrane is pre-treated with labelling particles which are activated with a reagent that specifically binds to the analyte of interest (if it is present in the sample) to form a complex. These complexes and/or unreacted labelling particles propagate along the membrane and arrive at a test region, which is pre-treated with an immobilized binding reagent that specifically binds to the analyte-bound complexes but does not bind non-complexed labelling particles. The labelling particles, which are immobilized in the test region if bound to the analyte of interest, have a distinctive colour or other detectable optical property. Therefore, the aggregation of labelling particles in the test region provides an observable indication that the analyte of interest is present in the tested sample.

Use of enzymes as detection reagents in analyte-specific diagnostics is also known and this approach forms the basis of the enzyme-linked immunosorbent assay (ELISA), which is considered to be a gold standard in laboratory-based analytics. The high sensitivity of ELISA is directly attributed to the enzyme reagent, since a single enzyme can generate many reporter molecules, thus amplifying the optical signal as compared to direct labelling with an optically detectable reporter. This enzymatic detection is typically a multi-step process, requiring each of the following: i) binding of the enzyme to the analyte of interest; ii) washing away excess, unbound enzyme and iii) addition of the enzyme-reactive substrate that is subsequently converted to a fluorescent or coloured product for detection.

Whilst lateral flow test strips such as those discussed above, which are a popular format for point-of-care sensing devices, provide a simple means for the automation of steps i) and ii) (i.e. the binding of a detection reagent and washing), the standard geometry of these devices does not enable the subsequent addition of a second solution containing the enzyme-reactive substrate without the need for additional user input. Since the majority of applications for lateral flow test devices are carried out by end users with either no or limited technical training, this requirement of a second user input or step after sample application to introduce the enzyme substrate may represent an unacceptable level of complexity and/or may result in inaccuracies within the test results as a consequence of user error. For these reasons, lateral flow test devices still primarily rely on the use of direct fluorescent or colorimetric detection reagents, despite there being a clear disadvantage to this approach in that a limited number of labelling particles can be immobilised for each molecule of analyte to be detected, which negatively impacts on device sensitivity for lower analyte concentrations.

In addition, and although the sensitivity limitation issue associated with the use of direct fluorescent or colorimetric detection reagents in lateral flow detection devices can be somewhat mitigated by use of high extinction coefficient optical reporters such as gold nanoparticles or quantum dots, the large size of these reporters increases their non-specific binding to the test line in the absence of analyte through London forces and other non-covalent interactions, which is another factor that is known to limit the sensitivity of lateral flow devices.

The use of enzymic detection reagents in combination with chemiluminescent substrates in lateral flow-based test devices to provide high sensitivity detection of nucleic acids, antibodies and proteins are disclosed in Ref. 1, Ref. 2 and Ref. 3, respectively. For example, Ref. 2 demonstrates that a 10× higher sensitivity can be achieved using enzymatic chemiluminescent detection in comparison to the use of a gold nanoparticle detection. However, none of the devices disclosed in any of these references is capable of automating the required subsequent delivery of an enzyme substrate once the excess of enzyme detection reagent has been washed off the test line. Instead, Ref. 1 and Ref. 2 each require manual delivery of a solution of the enzyme substrate solution onto the test line via a pipette, but this requires an additional user input which may well be unacceptable for point of care applications, particularly as the vast majority of lateral flow test devices are operated by unskilled or semi-skilled users in non-laboratory environments. In contrast to Ref. 1 and Ref. 2, the device disclosed in Ref. 3 represents a significant modification of the basic lateral flow device structure to better enable multistep analysis by including cross-flow strips across the main strip to deliver additional reagents to the test line after the initial analyte-binding event has occurred at the test line. However, this does not in any way automate the process and these additional reagents still needed to be manually transferred onto the cross-flow strip by a secondary user input after the conventional lateral flow step is complete.

In summary, and although the detection reagents used in Ref. 1, Ref. 2 and Ref. 3 are similar to those envisioned for use in the devices of the present invention, they are incorporated in such a way that a secondary user input, after sample application, is required to introduce the enzymatic substrate once the initial lateral flow step is complete. Therefore, the need for alternative analyte detection devices and methods, which utilise a conventional lateral flow device architecture but also enable the automation of the multiple steps needed for use of enzymatic detection reagents, is clear. In particular, the present invention utilizes enzyme detection reagents, which are relatively small in size and have lower non-specific binding but which can generate an equivalent or greater optical signal per analyte ratio than is generated using direct binding of high extinction coefficient reporters described above, in high sensitivity lateral flow test devices that do not add complexity to the existing lateral flow device construction, and can detect the presence of an analyte of interest in a single step, without the need for additional user input beyond standard sample application

### STATEMENTS OF THE INVENTION

According to a first aspect, there is provided an assay device for determining the presence of an analyte in a liquid sample, the device comprising:
a lateral flow membrane comprising a test region disposed between opposite first and second end regions of the membrane;
a fluid permeable layer having an area in contact with the first end region of the membrane; and
a sample pad in fluid communication with the fluid permeable layer for delivery of a liquid sample to the first region of the membrane via the fluid permeable layer, wherein:
   the fluid permeable layer comprises a mobile assay component configured to selectively bind the target analyte, the mobile assay component comprising a reporter enzyme which specifically converts an active substrate into an optical reporting molecule;
   the sample pad comprises a mobile latent substrate, the latent substrate comprising one or more active substrate precursor compounds that is/are not reactive with a reporter enzyme; and
   the test region comprises: an immobilized assay component for retaining the mobile assay component in the test region in dependence on the binding between the analyte, the mobile assay component and the immobilized assay component; and an immobilized activation enzyme that specifically converts the latent substrate into the active substrate.

The use of such a mobile latent substrate in combination with an immobilized activation enzyme allows the use of enzyme-based detection reagents in a lateral flow detection device that enables analyte detection in a single step, without the need for addition user input beyond sample application or alteration of conventional lateral flow device geometry. In particular, as the substrate is provided in a precursor form (the latent substrate) that is non-reactive with the reporter enzyme, the reporter enzyme and substrate do not need to be spatially or temporally separated as they pass along the lateral flow membrane towards the test region. The substrate is then enzymatically converted to its active form within the test region upon contact with the immobilized activation enzyme.

In preferred embodiments, the activation enzyme is glucose oxidase and the latent substrate comprises glucose. Alternatively, glucose oxidase and glucose can be replaced with any combination of an oxidase and its corresponding substrate provided that the selected oxidase and substrate react together to produce hydrogen peroxide as an active substrate. Numerous examples of such combinations are known and are commercially available.

Preferably, the latent substrate further comprises a compound selected from amplex red, o-phenylenediamine dihydrochloride, 3,3′,5,5′-tetramethylbenzidine, 2,2′-azino-di-[3-ethylbenzthiazoline-6-sulfonic acid], 3,3′-diaminobenzidine, 3-amino-9-ehtylcabazole luminol and combinations thereof. However, as an alternative or in addition to the inclusion in the latent substrate, the immobilized assay component may comprise a compound selected from amplex red, o-phenylenediamine dihydrochloride, 3,3′,5,5′-tetramethylbenzidine, 2,2′-azino-di-[3-ethylbenzthiazoline-6-sulfonic acid], 3,3′-diaminobenzidine, 3-amino-9-ehtylcabazole luminol and combinations thereof. These compounds, in the presence of reporter enzyme, react with the hydrogen peroxide active substrate to form a fluorescent, coloured or chemiluminescent optical reporting molecule.

Preferably, the reporter enzyme is a peroxidase enzyme, more preferably horseradish peroxidase or a polymer thereof. In the presence of horseradish peroxidase, the hydrogen peroxide of the active substrate reacts with the compound(s) identified above in a 1:1 stoichiometric ratio to generate the fluorescent, coloured or chemiluminescent optical reporting molecule. Therefore, this reaction will only proceed once a sufficient quantity of hydrogen peroxide has been generated through a reaction between the immobilized activation enzyme and the latent substrate dissolved or dispersed in the liquid test sample.

Preferably, the sample pad comprises latent substrate in an amount sufficiently high to ensure that, after delivery of the liquid sample to the sample pad, the concentration of latent substrate in the liquid sample is not the rate limiting factor for the activation enzyme-catalysed conversion of latent substrate to active substrate in the test region of the lateral flow membrane and, as a consequence, cannot be rate limiting for the subsequent reaction between the active substrate and the reporter enzyme.

In contrast to the latent substrate, which is typically provided in an excess in the sample pad, the fluid permeable layer preferably comprises the mobile assay component in an amount sufficiently low to ensure that it will be completely dissolved by the solvent front after delivery of the liquid sample to the sample pad. This ensures that primarily only the mobile assay components that are specifically immobilized via binding between the analyte, mobile assay component and immobilized assay component will be immobilized in the test region of the lateral flow membrane once the sample has passed through said test region.

In preferred embodiments, the mobile assay component comprises a conjugate of any analyte-specific biological receptor (e.g. antibody, nucleic acid aptamer, peptide aptamer, molecularly imprinted polymer, carbohydrate or a non-antibody binding protein) and the reporter enzyme. More preferably, the mobile assay component comprises a conjugate of an analyte-specific antibody or nucleic acid and the reporter enzyme. More preferably still, the mobile assay component comprises a conjugate of an analyte-specific antibody and the reporter enzyme. Similarly, the immobilized assay component preferably comprises an analyte-specific biological receptor, more preferably an antibody or nucleic acid, still more preferably an antibody, that is immobilized or tethered in the test region of the lateral flow membrane. These arrangements of mobile and immobilized assay components provide for a lateral flow device-based sandwich assay. However, the general concept of using a latent substrate disclosed herein can also be applied to other convention lateral flow device-based assay formats such as, for example, competitive binding assays.

In some embodiments, the test region of the lateral flow membrane comprises a test line on which the activation enzyme and immobilized assay component are co-immobilized. Alternatively, the activation enzyme may be immobilized on a first test line which is adjacent to a second test line on which the immobilized assay component is immobilized. In the latter arrangement, the first test line is preferably disposed between the first end region of the lateral flow membrane and the second test line to ensure that the latent substrate contacts the activation enzyme (and thus is converted into the active substrate) before passing the immobilized assay component. However, in a less preferred embodiment, said first test line may be disposed between the second end region of the lateral flow membrane and said second test line.

The device preferably further comprises an absorptive layer or wicking pad that is in fluid contact with the second region of the lateral flow membrane. The absorptive layer comprises one or more absorbent materials such as cotton linter or cellulose, and functions to draw the liquid sample through the lateral flow membrane by capillary flow and acts as a container for fluid that has passed through the test layer. Preferably the absorbent layer is capable of absorbing the entire volume of the applied liquid sample, so that it allows the entire volume of the sample to pass through the test region.

The device may be configured such that at least a portion of the test region of the lateral flow membrane is visible to the user. In such a configuration, any optical signal generated within the test region in dependence on the binding between the analyte, the mobile assay component and the immobilized assay component can be simply read by eye. However, for enhanced accuracy, the device may be configured such that any optical signal generated within the test region is readable by a camera and associated image processing software, or by an electronic reader comprising a light source (e.g. an organic light emitting diode) and an associated detector (e.g. an organic photodetector), which may be arranged in transmission or reflection mode.

According to an alternative aspect of the invention there is provided a test kit, the kit comprising the aforementioned device and a housing, wherein the device is disposed in the housing. Ideally, the housing comprises a base portion and a lid portion comprising first and second apertures, wherein the device is disposed in the housing such that at least a part of the sample pad is accessible through the first aperture and at least a part of the test region is readable/visible through the second aperture. Such an arrangement secures the device and covers parts of the device that do not require exposure. The housing is typically made from a polymer such as polycarbonate, polystyrene, polypropylene or similar.

According to yet another alternative aspect, the invention provides for the use of the aforementioned device or test kit to test for the presence of an analyte in a liquid sample. Preferably the liquid sample is selected from saliva, blood or urine.

According to yet another alternative aspect of the invention, there is provided a method for determining the presence of an analyte in a liquid sample, the method comprising: providing the aforementioned device or test kit; providing a liquid sample for analysis; and applying the liquid sample to the sample pad of said device. Preferably the liquid sample is selected from saliva, blood or urine.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following detailed description of certain embodiments. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

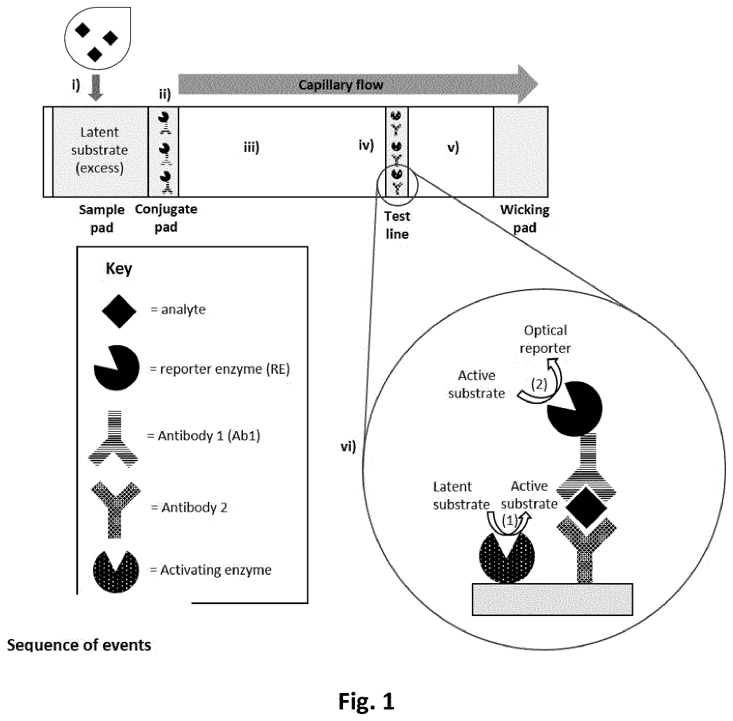

FIG. 1 is a schematic representation of an exemplary lateral flow device in accordance with the invention, which utilizes a sandwich assay for the single-step detection of an analyte within a liquid sample.

### DETAILED DESCRIPTION OF CERTAIN EMBODIMENTS

FIG. 1 provides a specific example of the use of a latent substrate-based test device of the invention in a sandwich assay to determine the presence of an analyte in a liquid test sample.

Shown is a device comprising: (i) a sample pad, which comprises an excess amount of latent substrate (denoted LS); (ii) a conjugate pad, which is a fluid permeable layer disposed between the sample pad and a lateral flow membrane, and which comprises a mobile assay component (denoted Ab1-RE), specifically a conjugate of reporter enzyme (e.g. horseradish peroxidase) and an analyte specific 1^{st} antibody; (iii) a lateral flow membrane that contains a test region disposed between opposite end regions of the membrane, the test region comprising a single test line on which the activation enzyme and an immobilized assay component (denoted Ab2), which is an analyte-specific 2^{nd} antibody; and (iv) an absorptive layer or wicking pad.

The following sequence of events show how such a device can be used to test for presence if an analyte within a liquid sample:
a liquid test sample suspected of containing the analyte of interest is first applied by the end user to the sample pad of the device, thereby dissolving an excess amount of LS within the liquid test sample;
the liquid sample (containing LS) passes through the conjugate pad, lateral flow membrane and into the wicking pad by capillary flow;
as the liquid sample passes into the conjugate pad, Ab1-RE conjugate is dissolved within the solvent front of the LS containing liquid sample;
if present, analyte within the liquid sample will bind to the Ab1-RE conjugate as the sample passes through the conjugate pad and across the lateral flow membrane towards the test line. No enzymic reactions take place at this stage, as no active substrate or activation enzyme is present within the sample;
as the liquid sample (containing LS, Ab1-RE and, if analyte is present, Ab1-RE/analyte complex) reaches the test line, any Ab1-RE/analyte complexes will bind to Ab2, becoming immobilized at the test line. In contrast, any non-complexed AB1-RE conjugates will continue to flow through the lateral flow membrane before arriving at the wicking pad;
a concerted series of enzymic reactions will take place at the test line, provided that AB1-RE/analyte complexes have bound to the immobilized Ab2 Specifically, LS is converted to active substrate by the immobilized activation enzyme, and the active substrate is then converted to form a fluorescent, coloured or chemiluminescent optical reporting molecule by the reporter enzyme of the immobilized Ab2/analyte/Ab1-RE complex, confirming the presence of analyte within the tested liquid sample.

As shown above, the present invention provides an enzyme-based lateral flow detection device having low non-specific binding and a high optical signal per analyte ratio, and which can detect the presence of an analyte of interest in a single step, without the need for additional user input beyond standard sample application.

### REFERENCES

1. Development of Chemiluminescent Lateral Flow Assay for the Detection of Nucleic Acids Wang Y., Fill C. and Nugen, S. R. Biosensors 2012, 2(1), 32-42; doi: 10.3390/bios2010032.

2. Moving Enzyme-Linked ImmunoSorbent Assay to the Point-of-Care Dry-Reagent Strip Biosensors

Kawde A. N., Mao X., Xu H., Zeng Q., He Y., and Liu G Am. J. Biomed. Sci. 2010, 2(1), 23-32; doi: 10.5099/aj100100023.

3. Chemiluminometric Immunosensor for High-Sensitivity Cardiac Troponin I Employing a Polymerized Enzyme Conjugate as a Tracer.

Lim G. S., Seo S. M., Paek S. H., Kim S. W., Jeon J. W., Kim D. H., Cho I. H., and Paek S. H. Sci Rep. 2015 5:14848; doi: 10.1038/srep14848.

1. An assay device for determining the presence of an analyte in a liquid sample, the device comprising:
a lateral flow membrane comprising a test region disposed between opposite first and second end regions of the membrane;
a fluid permeable layer having an area in contact with the first end region of the membrane; and
a sample pad in fluid communication with the fluid permeable layer for delivery of a liquid sample to the first region of the membrane via the fluid permeable layer, wherein:
the fluid permeable layer comprises a mobile assay component configured to selectively bind the target analyte, the mobile assay component comprising a reporter enzyme which specifically converts an active substrate into an optical reporting molecule;
the sample pad comprises a mobile latent substrate, the latent substrate comprising one or more active substrate precursor compounds that is/are not reactive with a reporter enzyme; and
the test region comprises: an immobilized assay component for retaining the mobile assay component in the test region in dependence on the binding between the analyte, the mobile assay component and the immobilized assay component; and an immobilized activation enzyme that specifically converts the latent substrate into the active substrate.

2. The device according to claim 1, wherein the activation enzyme is an oxidase enzyme and the latent substrate comprises a precursor compound that undergoes oxidation to hydrogen peroxide upon contact with the activation enzyme

3. The device according to claim 2, wherein the activation enzyme is glucose oxidase and the latent substrate comprises glucose as a precursor compound.

4. The device according to claim 1, wherein the latent substrate further comprises a compound selected from amplex red, o-phenylenediamine dihydrochloride, 3,3′,5,5′-tetram ethylb enzi dine, 2,2′-azino-di-[3-ethylbenzthiazoline-6-sulfonic acid], 3,3′-diaminobenzidine, 3-amino-9-ehtylcabazole luminol and combinations thereof.

5. The device according to claim 1, wherein the immobilized assay component comprises a compound selected from amplex red, o-phenylenediamine dihydrochloride, 3,3′,5,5′-tetram ethylb enzi dine, 2,2′-azino-di-[3-ethylbenzthiazoline-6-sulfonic acid], 3,3′-diaminobenzidine, 3-amino-9-ehtylcabazole luminol and combinations thereof.

6. The device according to claim 1, wherein the reporter enzyme is a peroxidase enzyme.

7. The device according to claim 6, wherein the reporter enzyme is a horseradish peroxidase or a polymer thereof.

8. The device according to claim 1, wherein the sample pad comprises latent substrate in an amount sufficiently high to ensure that, after delivery of the liquid sample to the sample pad, the concentration of latent substrate in the liquid sample is not the rate limiting factor for the activation enzyme-catalyzed conversion of latent substrate to active substrate.

9. The device according to claim 1, wherein the fluid permeable layer comprises the mobile assay component in an amount sufficiently low to ensure that it will be completely dissolved by the solvent front after delivery of the liquid sample to the sample pad.

10. The device according to claim 1, wherein the mobile assay component comprises a conjugate of an analyte-specific biological receptor and the reporter enzyme.

11. The device according to claim 10, wherein the mobile assay component comprises a conjugate of an analyte-specific antibody or nucleic acid and the reporter enzyme.

12. (canceled)

13. The device according to claim 1, wherein the immobilized assay component comprises an analyte-specific biological receptor that is immobilized in the test region of the lateral flow membrane.

14. The device according to claim 13, wherein the immobilized assay component comprises an analyte-specific antibody or nucleic acid that is immobilized in the test region of the lateral flow membrane.

15. (canceled)

16. The device according to claim 1, wherein the test region comprises a test line on which the activation enzyme and immobilized assay component are co-immobilized.

17. The device according to claim 1, wherein the test region comprises at least two test lines, the activation enzyme being immobilized on a first test line and the immobilized assay component being immobilized on a second test line.

18. The device according to claim 17, wherein the first test line is disposed between the first end region of the lateral flow membrane and the second test line.

19. The device according to claim 1, further comprising an absorptive layer in fluid contact with the second end region of the membrane.

20. A test kit comprising:
a device according to claim 1; and
a housing;
wherein the device is disposed in the housing.

21. (canceled)

22. The use of the device according to claim 1 or the test kit according to claim 20 to test for the presence of an analyte in a liquid sample.

23. (canceled)

24. A method for determining the presence of an analyte in a liquid sample, the method comprising:
providing a device according to claim 1 or a test kit according to claim 20;
providing a liquid sample for analysis; and
applying the liquid sample to the sample pad of said device.

25. (canceled)
